(19) United States
(12) Patent Application Publication
(10) Pub. No.: US 2024/0110211 A1
(43) Pub. Date: Apr. 4, 2024

(54) TRANSFORMED CELL HAVING ABILITY TO PRODUCE 2,5-PYRIDINE DICARBOXYLIC ACID

(71) Applicant: Kao Corporation, Chuo-ku, Tokyo (JP)

(72) Inventors: Kyoshiro NONAKA, Wakayama-Shi, Wakayama (JP); Fumikazu TAKAHASHI, Wakayama-Shi, Wakayama (JP)

(73) Assignee: Kao Corporation, Chuo-ku, Tokyo (JP)

(21) Appl. No.: 17/791,377

(22) PCT Filed: Dec. 28, 2020
(86) PCT No.: PCT/JP2020/049104

§ 371 Date: Jul. 7, 2022

(30) Foreign Application Priority Data
Jan. 8, 2020 (JP) 2020-001628

(51) Int. Cl. C12P 17/12; C12N 9/02; C12N 9/10; C12N 9/88; C12N 15/70; C12P 7/44

(52) U.S. Cl. CPC C12P 17/12; C12N 9/0069; C12N 9/1096; C12N 9/88; C12N 15/70; C12P 7/44; C12Y 113/11; C12Y 206/01; C12Y 401/03038; C12N 2800/101

(57) ABSTRACT

Provided are a transformed cell having the ability to produce 2,5-pyridine dicarboxylic acids, and a method for producing 2,5-pyridine dicarboxylic acids using the same. The present invention provides a transformed cell having the ability to produce 2,5-pyridine dicarboxylic acids, the transformed cell being derived from a microbe having the ability to biosynthesize 4-aminobenzoic acids and having the enhanced expression of the following polypeptides (I) and (II): (I) a polypeptide having 4-aminobenzoic acid hydroxylation activity, and (II) a polypeptide having 4-amino-3-hydroxybenzoate 2,3-dioxygenase activity.

### FIELD OF THE INVENTION

The present invention relates to a transformed cell having the ability to produce 2,5-pyridine dicarboxylic acids and use thereof.

### BACKGROUND OF THE INVENTION

The development of methods for producing compounds by biological approaches using renewable resources as starting materials is important for the realization of sustainable society. 2,5-Pyridine dicarboxylic acids (2,5-PDCAs) have a backbone similar to that of terephthalic acid which is produced in large amounts from nonrenewable resources as resin starting materials, and methods for producing 2,5-PDCAs by biological approaches from plant residues are also known. Hence, these compounds are expected as terephthalic acid alternatives obtainable from renewable resources (Patent Literature 1 and Non Patent Literatures 1 and 2). 2,5-Pyridine dicarboxylic acids are also known as precursors of nicotinic acid which is a water-soluble vitamin useful for food and pharmaceutical purposes, and further, also used as starting materials for plant growth promoters, organic light-emitting materials, adhesives, and the like (Patent Literature 2 and Non Patent Literatures 3 to 5).

A method of allowing a Rhodococcus jostii RHA1 strain harboring protocatechuate 2,3-dioxygenase to act on lignin is disclosed as a method for biologically producing 2,5-pyridine dicarboxylic acids (Patent Literature 1 and Non Patent Literature 2). However, its productivity is very low, and use of lignin, which nonuniformly contains many kinds of compounds, makes it difficult for this approach to stably produce 2,5-pyridine dicarboxylic acids. Furthermore, it is also necessary to separately add ammonium chloride as a nitrogen source necessary for reaction.

In addition, reaction mediated by 4-amino-3-hydroxybenzoate 2,3-dioxygenase (ahdA) carried by a Bordetella sp. 10d strain isolated as a 4-amino-3-hydroxybenzoic acid-utilizing bacterium is known as biological reaction to obtain 2,5-pyridine dicarboxylic acids (Non Patent Literature 6). According to this literature, it was confirmed in purified enzymes that 4-amino-3-hydroxybenzoic acid is oxidatively cleaved into 2-amino-5-carboxymuconic 6-semialdehyde through 4-amino-3-hydroxybenzoate 2,3-dioxygenase, followed by nonenzymatic self-conversion into 2,5-pyridine dicarboxylic acid.

However, in the Bordetella sp. 10d strain, 2-amino-5-carboxymuconic 6-semialdehyde is converted into 2-hydroxymuconic 6-semialdehyde through deaminase (ahdB). In actuality, no 2,5-pyridine dicarboxylic acid was detected in a culture solution, resulting in unsuccessful production of 2,5-pyridine dicarboxylic acids. If the production of 2,5-pyridine dicarboxylic acids is attempted using purified 4-amino-3-hydroxybenzoate 2,3-dioxygenase, it is necessary to add 4-amino-3-hydroxybenzoic acids as starting materials. Thus, it is difficult to produce 2,5-pyridine dicarboxylic acids practically from the economic standpoint.
[Patent Literature 1] WO 2016/202875
[Patent Literature 2] JP-A-2018-83789
[Non Patent Literature 1] Alessandro Pellis et al., Nature Communications, Vol. 10, pp. 1762 (2019)
[Non Patent Literature 2] Zoe Mycroft et al., Green Chemistry, Vol. 17, pp. 4974 (2015)
[Non Patent Literature 3] Erick J. Vandamme, Jose Luis Revuelta, Industrial Biotechnology of Vitamins, Biopigments, and Antioxidants (Book), John Wiley & Sons, 2016
[Non Patent Literature 4] Min Zeng et al., Journal of Materials Chemistry C, Vol. 7, pp. 2751 (2019)
[Non Patent Literature 5] Qiao Zhang et al., Journal of the American Chemical Society Vol. 141, pp 8058 (2019)
[Non Patent Literature 6] Shinji Takenaka et al., European Journal of Biochemistry, Vol. 269, pp. 5871 (2002)

### SUMMARY OF THE INVENTION

The present invention relates to the following 1) to 2).
1) A transformed cell having ability to produce 2,5-pyridine dicarboxylic acids, the transformed cell being derived from a microbe having ability to biosynthesize 4-aminobenzoic acids and having enhanced expression of the following polypeptides (I) and (II):
(I) a polypeptide having 4-aminobenzoic acid hydroxylation activity, and
(II) a polypeptide having 4-amino-3-hydroxybenzoate 2,3-dioxygenase activity.
2) A method for producing 2,5-pyridine dicarboxylic acids or salts thereof, comprising a step of culturing the transformed cell according to 1).

### DETAILED DESCRIPTION OF THE INVENTION

The present invention relates to provision of a transformed cell having the ability to produce 2,5-pyridine dicarboxylic acids, and a method for producing 2,5-pyridine dicarboxylic acids using the same.

The present inventors prepared a microbe producing a polypeptide having particular enzyme activity and found that 2,5-pyridine dicarboxylic acids can be efficiently produced using this microbe.

According to the present invention, 2,5-pyridine dicarboxylic acids for use as starting materials for resins, foods, medicaments, plant growth promoters, organic light-emitting materials, adhesives, and the like can be efficiently produced.

In the present invention, the identity of an amino acid sequence or a nucleotide sequence is calculated by the Lipman-Pearson method (Science, 1985, 227: 1435-1441). Specifically, the identity is calculated by analysis with a unit size to compare (ktup) set to 2 using the homology analysis (search homology) program of genetic information processing software GENETYX Ver. 12.

In the present invention, the term “at least 90% identity” in relation to an amino acid sequence or a nucleotide sequence refers to 90% or higher, preferably 95% or higher, more preferably 96% or higher, even more preferably 97% or higher, even more preferably 98% or higher, even more preferably 99, or higher identity.

In the present invention, the term “amino acid sequence derived by having the deletion, substitution, addition, or insertion of one or more amino acids” refers to an amino acid sequence having the deletion, substitution, addition, or insertion of 1 or more and 10 or less, preferably 1 or more and 8 or less, more preferably 1 or more and 5 or less, even more preferably 1 or more and 3 or less amino acids. In the present specification, the term “nucleotide sequence having the deletion, substitution, addition, or insertion of one or more nucleotides” refers to a nucleotide sequence having the deletion, substitution, addition, or insertion of 1 or more and 30 or less, preferably 1 or more and 24 or less, more preferably 1 or more and 15 or less, even more preferably 1 or more and 9 or less nucleotides. In the present specification, the “addition” of an amino acid or a nucleotide includes the addition of an amino acid or a nucleotide to one or both ends of a sequence.

In the present invention, the “operable linkage” of a gene to a control region means that the gene is linked to the control region such that the gene is expressible under the control of the control region. The procedures of the “operable linkage” of a gene to a control region are well known to those skilled in the art.

In the present invention, the term “original” which is used for a function, property, or trait of a cell is used for indicating that the function, the property, or the trait is present in the wild type of the cell. By contrast, the term “foreign” is used for indicating that the function, the property, or the trait is introduced ab extra, not indigenous to the cell. For example, a “foreign” gene or polynucleotide is a gene or a polynucleotide introduced ab extra into a cell. The foreign gene or polynucleotide may be derived from an organism of the same species as that of the cell harboring it or may be derived from an organism of different species therefrom (i.e., a heterologous gene or polynucleotide).

In the present invention, specific examples of the “2,5-pyridine dicarboxylic acids” include 2,5-pyridine dicarboxylic acid derivatives of the following formula (1):

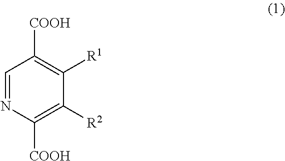

wherein R¹ and R² each independently represent a hydrogen atom, a hydroxy group (—OH), a methoxy group (—OCH₃), an amino group (—NH₂), a halogen atom, a carboxy group (—COOH), a methyl group (—CH₃), or an ethyl group (—CH₂CH₃).

The 2,5-pyridine dicarboxylic acid derivatives can be present in the forms of salts. Examples of such salts include salts with alkali metals such as sodium and potassium, and salts with alkaline earth metals such as calcium and magnesium.

Specific examples of the 4-aminobenzoic acids include 4-aminobenzoic acid derivatives of the following formula (2):

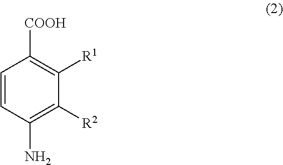

wherein R¹ and R² are as defined above.

In the formula (1) or (2), examples of the halogen atom represented by R¹ or R² include a fluorine atom, a chlorine atom, a bromine atom, and an iodine atom. A fluorine atom is preferred.

Each of R¹ and R² is preferably a hydrogen atom, a hydroxy group (—OH), a methoxy group (—OCH₃), a fluorine atom (—F) or a methyl group (—CH₃). More preferably, both R¹ and R² are hydrogen atoms.

In the present invention, the “transformed cell having the ability to produce 2,5-pyridine dicarboxylic acids” is a microbial cell which is derived from a microbe having the ability to biosynthesize 4-aminobenzoic acids, and has the enhanced expression of (I) a polypeptide having 4-aminobenzoic acid hydroxylation activity, and (II) a polypeptide having 4-amino-3-hydroxybenzoate 2,3-dioxygenase activity.

Examples of the (I) polypeptide having 4-aminobenzoic acid hydroxylation activity include (A) a polypeptide which consists of the amino acid sequence represented by SEQ ID NO: 2, or a polypeptide which consists of an amino acid sequence having at least 90% identity to the amino acid sequence represented by SEQ ID NO: 2, and has 4-aminobenzoic acid hydroxylation activity, and (B) a polypeptide which consists of the amino acid sequence represented by SEQ ID NO: 4, or a polypeptide which consists of an amino acid sequence having at least 90%. identity to the amino acid sequence represented by SEQ ID NO: 4, and has 4-aminobenzoic acid hydroxylation activity.

In this context, the polypeptide which consists of the amino acid sequence represented by SEQ ID NO: 2 (also referred to as “HFM122”) and the polypeptide which consists of the amino acid sequence represented by SEQ ID NO: 4 (also referred to as “HFM689”) are each known as 4-hydroxybenzoate 3-monooxygenase (EC1.14.13.2). The 4-hydroxybenzoate 3-monooxygenase is an enzyme having catalytic activity of accelerating any one or both of reaction to produce protocatechuic acid by hydroxylating position 3 of 4-hydroxybenzoic acid and inverse reaction thereof, and is an enzyme which catalyzes the hydroxylation of 4-hydroxybenzoic acids (4-hydroxybenzoate hydroxylase).

The 4-hydroxybenzoate 3-monooxygenase is known to have activity of catalyzing the hydroxylation of position 3 of its original substrate 4-hydroxybenzoic acid as well as 4-aminobenzoic acid having a molecular structure similar thereto (e.g., Domenico L. Gatti et al., Biochemistry, Vol. 35, No. 2, pp. 567-578 (1996)). The present applicant found that these enzymes HFM122 and HFM689 have activity of catalyzing the hydroxylation of 4-aminobenzoic acids, preferably activity of catalyzing the hydroxylation of position 3 of 4-aminobenzoic acids (Japanese Patent Application No. 2018-171849). Thus, in the present invention, the “4-aminobenzoic acid hydroxylation activity” means activity of catalyzing the hydroxylation of 4-aminobenzoic acids, preferably activity of catalyzing the hydroxylation of position 3 of 4-aminobenzoic acids.

This 4-aminobenzoic acid hydroxylation activity can be determined by, for example, a method described in Japanese Patent Application No. 2018-171849.

Examples of the amino acid sequence having at least 90% identity to the amino acid sequence represented by SEQ ID NO: 2 or 4 include an amino acid sequence consisting of the amino acid sequence represented by SEQ ID NO: 2 or 4 but having the deletion, substitution, addition, or insertion of one or more amino acids.

Examples of the (II) polypeptide having 4-amino-3-hydroxybenzoate 2,3-dioxygenase activity include (C) a polypeptide which consists of the amino acid sequence represented by SEQ ID NO: 6, or a polypeptide which consists of an amino acid sequence having at least 90% identity to the amino acid sequence represented by SEQ ID NO: 6, and has 4-amino-3-hydroxybenzoate 2,3-dioxygenase activity.

The polypeptide which consists of the amino acid sequence represented by SEQ ID NO: 6 is known as 4-amino-3-hydroxybenzoate 2,3-dioxygenase. The 4-amino-3-hydroxybenzoate 2,3-dioxygenase is an enzyme which oxidatively cleaves the benzene ring of 4-amino-3-hydroxybenzoic acid to form 2-amino-5-carboxymuconic 6-semialdehyde. The 4-amino-3-hydroxybenzoate 2,3-dioxygenase activity can be determined by, for example, a method known in the art (Non Patent Literature 6 described above)).

Examples of the amino acid sequence having at least 90% identity to the amino acid sequence represented by SEQ ID NO: 6 include an amino acid sequence consisting of the amino acid sequence represented by SEQ ID NO: 6 but having the deletion, substitution, addition, or insertion of one or more amino acids.

Examples of a method for introducing a mutation such as deletion, substitution, addition, or insertion of an amino acid to the amino acid sequence of any of the polypeptides include a method of introducing a mutation such as deletion, substitution, addition, or insertion of a nucleotide to a nucleotide sequence encoding the amino acid sequence. Examples of an approach for introducing a mutation to the nucleotide sequence include mutagenesis with a chemical mutagen such as ethyl methanesulfonate, N-methyl-N-nitrosoguanidine, or nitrous acid or with a physical mutagen such as ultraviolet ray, X ray, gamma ray, or ion beam, site-directed mutagenesis, and a method described by Dieffenbach et al. (Cold Spring Harbor Laboratory Press, New York, 581-621, 1995). Examples of the site-directed mutagenesis approach include a method using splicing overlap extension (SOE) PCR (Horton et al., Gene 77, 61-68, 1989), ODA method (Hashimoto-Gotoh et al., Gene, 152, 271-276, 1995), and Kunkel method (Kunkel, T. A., Proc. Natl. Acad. Sci. USA, 1985, 82, 488). Alternatively, a commercially available kit for site-directed mutagenesis such as Site-Directed Mutagenesis System Mutan-Super Express Km kit (Takara Bio Inc.), Transformer™ Site-Directed Mutagenesis kit (Clontech Laboratories, Inc.), or KOD-Plus-Mutagenesis Kit (Toyobo Co., Ltd.) may be used.

In the present invention, the transformed cell having the enhanced expression of the polypeptide having 4-aminobenzoic acid hydroxylation activity, and the polypeptide having 4-amino-3-hydroxybenzoate 2,3-dioxygenase activity can be a host cell containing, in an expressible state, polynucleotides necessary for the expression of the polypeptides. The polypeptides may be foreign ones or may be the original ones of the cell.

Examples thereof include cells expressibly harboring the polynucleotides, and cells having enhanced degrees of expression of the polynucleotides. Specific examples thereof include cells harboring vectors or DNA fragments containing the polynucleotides and control regions operably linked thereto, and cells having strong control regions replaced for the control regions of the polynucleotides.

In this context, for the polynucleotides, examples of (i) a polynucleotide encoding the polypeptide having 4-aminobenzoic acid hydroxylation activity include the following polynucleotides (a) and (b), and examples of (ii) a polynucleotide encoding the polypeptide having 4-amino-3-hydroxybenzoate 2,3-dioxygenase activity include the following polynucleotide (c) (these polynucleotides are also collectively referred to as the “polynucleotide of the present invention”):
(a) a polynucleotide which consists of the nucleotide sequence represented by SEQ ID NO: 1, or a polynucleotide which consists of a nucleotide sequence having at least 90%. identity to the nucleotide sequence represented by SEQ ID NO: 1, and encodes the polypeptide having 4-aminobenzoic acid hydroxylation activity;
(b) a polynucleotide which consists of the nucleotide sequence represented by SEQ ID NO: 3, or a polynucleotide which consists of a nucleotide sequence having at least 90% identity to the nucleotide sequence represented by SEQ ID NO: 3, and encodes the polypeptide having 4-aminobenzoic acid hydroxylation activity; and
(c) a polynucleotide which consists of the nucleotide sequence represented by SEQ ID NO: 5, or a polynucleotide which consists of a nucleotide sequence having at least 90% identity to the nucleotide sequence represented by SEQ ID NO: 5, and encodes the polypeptide having 4-amino-3-hydroxybenzoate 2,3-dioxygenase activity.

Examples of the nucleotide sequence having at least 90% identity to the nucleotide sequence represented by SEQ ID NO: 1, 3 or 5 include a nucleotide sequence consisting of the nucleotide sequence represented by SEQ ID NO: 1, 3 or 5 but having the deletion, substitution, addition, or insertion of one or more nucleotides. The method for introducing a mutation such as deletion, substitution, addition, or insertion of a nucleotide to the nucleotide sequence is as mentioned above. The polynucleotide may be in a single-stranded or double-stranded form, or may be DNA or RNA. The DNA may be cDNA or artificial DNA such as chemically synthesized DNA.

The polynucleotide may be incorporated into a vector. Preferably, the vector containing the polynucleotide of the present invention is an expression vector. Preferably, the vector is an expression vector which can transfer the polynucleotide of the present invention into a host microbe and enables the polynucleotide to be expressed within the host microbe. Preferably, the vector contains the polynucleotide of the present invention, and a control region operably linked thereto. The vector may be a vector capable of proliferating and replicating autonomously outside the chromosome (i.e., in a plasmid), or may be a vector which is incorporated into the chromosome.

Specific examples of the vector include pBluescript II SK(−) (Stratagene California), pUC series vectors such as pUC18/19 and pUC118/119 (Takara Bio Inc.), pET series vectors (Takara Bio Inc.), pGEX series vectors (GE Healthcare Japan Corp.), pCold series vectors (Takara Bio Inc.), pHY300PLK (Takara Bio Inc.), pUB110 (Mckenzie, T. et al., 1986, Plasmid 15 (2): 93-103), pBR322 (Takara Bio Inc.), pRS403 (Stratagene California), pMW218/219 (Nippon Gene Co., Ltd.), pRI series vectors such as pRI909/910 (Takara Bio Inc.), pBI series vectors (Clontech Laboratories, Inc.), IN3 series vectors (Inplanta Innovations Inc.), pPTR1/2 (Takara Bio Inc.), pDJB2 (D. J. Ballance et al., Gene, 36, 321-331, 1985), pAB4-1 (van Hartingsveldt W et al., Mol Gen Genet, 206, 71-75, 1987), pLeu4 (M. I. G. Roncero et al., Gene, 84, 335-343, 1989), pPyr225 (C. D. Skory et al., Mol Genet Genomics, 268, 397-406, 2002), and pFG1 (Gruber, F. et al., Curr Genet, 18, 447-451, 1990).

The polynucleotide may be constructed as a DNA fragment containing the same. Examples of the DNA fragment include PCR-amplified DNA fragments and restriction enzyme-cleaved DNA fragments. Preferably, the DNA fragment can be an expression cassette containing the polynucleotide of the present invention, and a control region operably linked thereto.

The control region contained in the vector or the DNA fragment is a sequence for allowing the polynucleotide of the present invention to be expressed within a host cell into which the vector or the DNA fragment has been introduced. Examples thereof include expression regulation regions such as promoters and terminators, and replication origins. The type of the control region can be appropriately selected according to the type of the host microbe into which the vector or the DNA fragment is introduced. If necessary, the vector or the DNA fragment may further have a selective marker such as an antibiotic resistance gene or an amino acid synthesis-related gene.

A general genetic transformation method, for example, electroporation, transformation, transfection, conjugation, protoplast method, particle gun method, or Agrobacterium method, can be used for transferring the vector or the DNA fragment into the host cell.

The transformed cell harboring the vector or the DNA fragment of interest can be selected by using the selective marker. When the selective marker is, for example, an antibiotic resistance gene, the cell harboring the vector or the DNA fragment of interest can be selected by culture in a culture medium supplemented with the antibiotic. When the selective marker is, for example, an amino acid synthesis-related gene, the cell harboring the vector or the DNA fragment of interest can be selected by using the presence or absence of the amino acid auxotrophy as an index after gene transfer into the host cell requiring the amino acid. Alternatively, the transfer of the vector or the DNA fragment of interest may be confirmed by examining the DNA sequence of the transformed cell by PCR or the like.

Examples of the strong control region include, but are not particularly limited to, high-expression promoters known in the art such as T7 promoter, lac promoter, tac promoter, trp promoter, tu promoter, and gap promoter.

Examples of the method for replacing the strong control region for the control region of the polynucleotide present on the genome of the host cell include a method of introducing a DNA fragment containing the polynucleotide sequences of the strong control region and a selective marker into the host cell, and selecting a transformed cell by homologous recombination or nonhomologous recombination.

In the present invention, a microbe having the ability to biosynthesize 4-aminobenzoic acids is used as the host cell. Any of cells of a fungi, a yeast, actinomycete, E. coli, Bacillus subtilis, or the like may be used as the microbe, and E. coli or actinomycete is preferred. Examples of the actinomycete include bacteria of the genus Corynebacterium, bacteria of the genus Mycobacterium, bacteria of the genus Rhodococcus, bacteria of the genus Streptomyces, and bacteria of the genus Propionibacterium. A bacterium of the genus Corynebacterium is preferred, and Corynebacterium glutamicum is more preferred.

The phrase “having the ability to biosynthesize 4-aminobenzoic acids” means that the microbe needs only to be able to supply 4-aminobenzoic acids. A microbe having enhanced ability to supply 4-aminobenzoic acids is more preferred. Examples of the method for enhancing the ability of the microbe to supply 4-aminobenzoic acids include a method of introducing, into a cell, a vector containing a polynucleotide encoding a polypeptide necessary for biosynthesizing 4-aminobenzoic acids, and a control region operably linked thereto, and a method of substituting the control region of a polynucleotide encoding a polypeptide necessary for biosynthesizing 4-aminobenzoic acids, which is originally carried by a cell, with a strong control region.

In this context, examples of the polypeptide necessary for biosynthesizing 4-aminobenzoic acids include (III) 4-amino-4-deoxychorismate synthase and (IV) 4-amino-4-deoxychorismate lyase. Thus, examples of the enhanced ability to supply 4-aminobenzoic acids include an aspect in which the expression of any one or more of these enzymes is enhanced.

Specific examples thereof include a method of introducing a vector or a DNA fragment containing one or more polynucleotides selected from the group consisting of (iii) a polynucleotide (e.g., SEQ ID NO: 7) encoding the 4-amino-4-deoxychorismate synthase and (iv) a polynucleotide (e.g., SEQ ID NO: 8) encoding the 4-amino-4-deoxychorismate lyase, and a control region operably linked thereto into a microbe, and a method of replacing a strong control region for one or more control regions of any of the following polynucleotides, which are originally carried by the microbe.

The method for introduction of the vector containing the polynucleotide of interest and the method for replacement of a strong control region for a control region can be performed in the same manner as the methods described above.

The transformed cell of the present invention thus prepared is cultured and evaluated for its productivity of 2,5-pyridine dicarboxylic acids, and a proper transformed cell can be selected to obtain a strain producing useful 2,5-pyridine dicarboxylic acids.

Methods for measuring products can be performed in accordance with methods described in Reference Examples mentioned later.

The method for producing 2,5-pyridine dicarboxylic acids or salts thereof according to the present invention is carried out by culturing the transformed cell mentioned above in a proper culture medium. The culture conditions can be appropriately designed depending on the microbe used.

Any of a natural culture medium and a synthetic culture medium may be used as the culture medium for the culture of the transformed cell as long as the culture medium contains a carbon source, a nitrogen source, inorganic salts, etc. and permits efficient culture of the transformed cell of the present invention. For example, saccharides such as glucose, polyols such as glycerin, alcohols such as ethanol, or organic acids such as pyruvic acid, succinic acid or citric acid can be used as carbon sources. Preferred examples thereof include saccharides such as glucose, maltose, sucrose, fructose, and materials containing them.

For example, peptone, meat extracts, yeast extracts, casein hydrolysates, alkali extracts of soymeal, alkylamines such as methylamine, or ammonia or its salt can be used as nitrogen sources.

In addition, phosphate, carbonate, sulfate, salts of magnesium, calcium, potassium, iron, manganese, zinc, or the like, a particular amino acid, a particular vitamin, an antifoaming agent, etc. may be used, if necessary.

The culture can usually be performed, at 10° C. to 40° C. for 6 hours to 72 hours, preferably for 9 hours to 60 hours, more preferably for 12 hours to 48 hours, if necessary, with stirring or shaking. During the culture, an antibiotic such as ampicillin or kanamycin may be added, if necessary, to the culture medium.

The methods for collecting and purifying 2,5-pyridine dicarboxylic acids from the cultures are not particularly limited. Specifically, the collection and the purification can be carried out by combining a well-known ion-exchange resin method, precipitation method, crystallization method, recrystallization method, concentration method and other methods. For example, after removal of bacterial cells by centrifugation or the like, ionic substances are removed with cation- and anion-exchange resins, and the resultant can be concentrated to obtain 2,5-pyridine dicarboxylic acids. 2,5-Pyridine dicarboxylic acids accumulated in the cultures may be used directly without being isolated.

In relation to the embodiments mentioned above, the present invention further discloses the following aspects.
<1> A transformed cell having ability to produce 2,5-pyridine dicarboxylic acids, the transformed cell being derived from a microbe having ability to biosynthesize 4-aminobenzoic acids and having enhanced expression of the following polypeptides (I) and (II):
(I) a polypeptide having 4-aminobenzoic acid hydroxylation activity, and
(II) a polypeptide having 4-amino-3-hydroxybenzoate 2,3-dioxygenase activity.
<2> The transformed cell according to <1>, wherein the (I) polypeptide having 4-aminobenzoic acid hydroxylation activity is (A) a polypeptide which consists of the amino acid sequence represented by SEQ ID NO: 2, or a polypeptide which consists of an amino acid sequence having at least 90% identity to the amino acid sequence represented by SEQ ID NO: 2, and has 4-aminobenzoic acid hydroxylation activity, or (B) a polypeptide which consists of the amino acid sequence represented by SEQ ID NO: 4, or a polypeptide which consists of an amino acid sequence having at least 90% identity to the amino acid sequence represented by SEQ ID NO: 4, and has 4-aminobenzoic acid hydroxylation activity.
<3> The transformed cell according to <1> or <2>, wherein the (II) polypeptide having 4-amino-3-hydroxybenzoate 2,3-dioxygenase activity is (C) a polypeptide which consists of the amino acid sequence represented by SEQ ID NO: 6, or a polypeptide which consists of an amino acid sequence having at least 90% identity to the amino acid sequence represented by SEQ ID NO: 6, and has 4-amino-3-hydroxybenzoate 2,3-dioxygenase activity.
<4> The transformed cell according to any of <1> to <3>, wherein the microbe having the ability to biosynthesize 4-aminobenzoic acids is a microbe having enhanced expression of one or more enzymes selected from the group consisting of the following (III) and (IV):
(III) 4-amino-4-deoxychorismate synthase, and
(IV) 4-amino-4-deoxychorismate lyase.
<5> The transformed cell according to any of <1> to <4>, wherein the transformed cell is E. coli or a bacterium of the genus Corynebacterium.
<6> The transformed cell according to <5>, wherein the bacterium of the genus Corynebacterium is Corynebacterium glutamicum.
<7> A method for producing 2,5-pyridine dicarboxylic acids or salts thereof, comprising a step of culturing a transformed cell according to any of <1> to <6>.
<8> The method according to <7>, wherein the culture is performed in a culture medium containing a saccharide as a carbon source.
<9> The method according to <7> or <8>, further comprising a step of collecting 2,5-pyridine dicarboxylic acids or salts thereof from the cultures.
<10> The method according to any of <6> to <8>, wherein the 2,5-pyridine dicarboxylic acids are 2,5-pyridine dicarboxylic acid derivatives of the following formula (1):

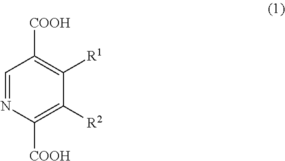

wherein R¹ and R² each independently represent a hydrogen atom, a hydroxy group, a methoxy group, an amino group, a halogen atom, a carboxy group, a methyl group, or an ethyl group, and
the 4-aminobenzoic acids are 4-aminobenzoic acid derivatives of the following formula (2):

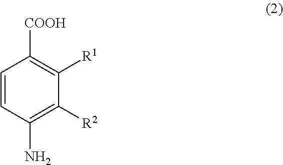

wherein R¹ and R² are as defined above.
<11> The method according to <10>, wherein in the formulas (1) and (2), both R¹ and R² are hydrogen atoms.

### EXAMPLES

Hereinafter, the present invention will be described in more detail with reference to Examples. However, the present invention is not limited thereto.

### Example 1 Production of 2,5-pyridine dicarboxylic acid

In the following Example, PCR was performed using PrimeSTAR Max Premix (Takara Bio Inc.), unless otherwise specified.

### (1) Preparation of plasmid for 2,5-pyridine dicarboxylic acid Production

### (a) Preparation of Plasmid pECsf_gapS_pabABC

A DNA fragment containing a gene encoding 4-amino-4-deoxychorismate synthase and a gene encoding 4-amino-4-deoxychorismate lyase was amplified by PCR using the genome extracted from a Corynebacterium glutamicum ATCC13032 strain by a routine method as a template and using primers GN14_127 (SEQ ID NO: 9, TATTAATTAAATGCGCGTTTTAATTATTGATAATTATGATTC) and GN14_133 (SEQ ID NO: 10, TTGCGGCCGCTTGTTTAAACCTCCTTACAGAAAAATGGTTGGGCG). This fragment was inserted between the PacI site and the NotI site of a plasmid pECsf_gapS (see JP-A-2016-146779) to obtain a plasmid pECsf_gapS_pabABC.

### (b) Preparation of Plasmid pECsf_gapS_pabABC HFM122

A DNA fragment for a vector was synthesized by PCR using the plasmid pECsf_gapS_pabABC obtained above as a template and using primers pabABCcory vec R (SEQ ID NO: 11, AAATTTAAACCTCCTTTACAGAAAAATGGTTGG) and pabABCcory vec F (SEQ ID NO: 12, GGAGGTTTAAACAAGCGGCCGCGATATC). Subsequently, a plasmid containing a gene (SEQ ID NO: 1) encoding a polypeptide HFM122 having 4-aminobenzoic acid hydroxylation activity was prepared by artificial gene synthesis, and a DNA fragment for an insert was synthesized by PCR using this plasmid as a template and using primers pECsfD HFM122 F (SEQ ID NO: 13, AGGAGGTTTAAATTTATGCGCACTCAGGTGGCTAT) and pECsfD HFM122 R (SEQ ID NO: 14, CTTGTTTAAACCTCCTTATACGAGTGGCAGTCCTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC HFM122. An ECOS Competent E. coli DH5a strain (Nippon Gene Co., Ltd.) was transformed with the obtained plasmid solution. The cell solution was spread over LBKm agar medium (1% Bacto Tryptone, 0.5% yeast extract, 1% NaCl, 50 μg/mL kanamycin sulfate, 1.5% agar) and then left standing overnight at 37° C. The obtained colonies were subjected to PCR reaction using Sapphire Amp (Takara Bio Inc.) and primers pabABC+pobA for CPCR F (SEQ ID NO: 15, GCTATCAAAACATTCGGCACATTGGTTTTCC) and pabABC+pobA for CPCR R (SEQ ID NO: 16, GGAAGATGCGTGATCTGATCCTTCAACTC) to select a transformant confirmed to harbor the DNA fragment of interest. The obtained transformant was inoculated to 2 mL of LBKm liquid medium (1% Bacto Tryptone, 0.5% yeast extract, 1% NaCl, 50 μg/mL kanamycin sulfate) and cultured overnight at 37° C. A plasmid was purified from this culture solution using NucleoSpin Plasmid EasyPure (Takara Bio Inc.).

### (c) Preparation of Plasmid

pECsf_gapS_pabABC_tuD_HFM122 A DNA fragment for a vector was synthesized by PCR using the plasmid pECsf_gapS_pabABC HFM122 obtained above as a template and using primers pabC last R (SEQ ID NO: 17, TTACAGAAAAATGGTTGGGCGCAA) and HFM122 F (SEQ ID NO: 18, ATGCGCACTCAGGTGGCTATCG). Subsequently, a DNA fragment (SEQ ID NO: 19, TACGTACCTGCAGGTAGCGTGTCAGTAGGCGCGTAGGGTAAGTGGGGTAGCGGCTTG TTAGATATCTTGAAATCGGCTTTCAACAGCATTGATTTCGATGTATTTAGCTGGCCG TTACCCTGCGAATGTCCACAGGGTAGCTGGTAGTTTGAAAATCAACGCCGTTGCCCT TAGGATTCAGTAACTGGCACATTTTGTAATGCGCTAGATCTGTGTGCTCAGTCTTCC AGGCTGCTTATCACAGTGAAAGCAAAACCAATTCGTGGCTGCGAAAGTCGTAGCCAC CACGAAGTCCAAAGGAGGATCTAAATTATGAATAATATAAAAGGAGGAATTAATTAA) containing tuf gene (cg0587) promoter (hereinafter, referred to as tu promoter) carried by a Corynebacterium glutamicum ATCC13032 strain was prepared by artificial gene synthesis, and a DNA fragment for an insert was synthesized by PCR using this fragment as a template and using primers pabC-Ptu F (SEQ ID NO: 20, ACCATTTTTCTGTAATACGTACCTGCAGGTAGCGTG) and Ptu-HFM122 R (SEQ ID NO: 21, CACCTGAGTGCGCATTTAATTAATTCCTCCTTTTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tuD_HFM122. An ECOS Competent E. coli DH5a strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire A.mp (Takara Bio Inc.) and primers Ptu seq 1 (SEQ ID NO: 22, GCTTGTTAGATATCTTGAAATCGGCTTTC) and pabABC+pobA for CPCR R (SEQ ID NO: 16, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

### (d) Preparation of Plasmid pECsf_gapS_pabABC_tu_HFM122

A DNA fragment for a vector was synthesized by PCR using the plasmid pECsf_gapS_pabABC_tuD_HFM122 obtained above as a template and using primers pGapABA_tu vec F (SEQ ID NO: 23, GGAGGTTTAAACAAGCGG) and pGapABA_tu vec R (SEQ ID NO: 24, AATTTAGATCCTCCTTTGGACTTCGTG). Subsequently, a DNA fragment for an insert was synthesized by PCR using a plasmid containing an HFM122-encoding gene (SEQ ID NO: 1) prepared by artificial gene synthesis as a template and using primers HFM122 ins F (SEQ ID NO: 25, AGGAGGATCTAAATTATGCGCACTCAGGTGGCTATC) and HFM122 ins R (SEQ ID NO: 26, CTTGTTTAAACCTCCTTATACGAGTGGCAGTCCTACG). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tu_HFM122. An ECOS Competent E. coli DH5a strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 22, GCTTGTTAGATATCTTGAAATCGGCTTTC) and pabABC+pobA for CPCR R (SEQ ID NO: 16, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

### (e) Preparation of Plasmid pECsf_gapS_pabABC_tu_HFM122_ahdA

A DNA fragment for a vector was synthesized by PCR using the plasmid pECsf_gapS_pabABC_tu_HFM122 obtained above as a template and using primers ahdA vec R (SEQ ID NO: 27, CGCTTGTTTAAACCTCCTTATACGAGTGGCAGTCCTACG) and ahdA vec F (SEQ ID NO: 28, GCCGCGATATCGTTGTAAAAAACCCCGCTC). Subsequently, a plasmid containing gene ahdA (SEQ ID NO: 5) encoding a polypeptide having 4-amino-3-hydroxybenzoate 2,3-dioxygenase activity was prepared by artificial gene synthesis, and a DNA fragment for an insert was synthesized by PCR using this plasmid as a template and using primers ahdA ins F (SEQ ID NO: 29, AGGTTTAAACAAGCGATGATCATCCTGGAAAACTTCAAGATG) and ahdA ins R (SEQ ID NO: 30, CAACGATATCGCGGCTTAATCGCGTCCTGGAGCAAC). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tu_HFM122 ahdA. An ECOS Competent E. coli DHSa strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 22, GCTTGTTAGATATCTTGAAATCGGCTTTC) and pabABC+pobA for CPCR R (SEQ ID NO: 16, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmid, the gene encoding 4-amino-4-deoxychorismate synthase and the gene encoding 4-amino-4-deoxychorismate lyase were linked under the control of gap promoter, and the gene encoding the polypeptide having 4-aminobenzoic acid hydroxylation activity and the gene encoding 4-amino-3-hydroxybenzoate 2,3-dioxygenase were further linked under the control of the tu promoter.

### (2) Transfer of Plasmid into Host Cell

A Corynebacterium glutamicum DRHG145 strain (see Japanese Patent Application No. 2014-523757) was transformed with a plasmid pECsf_gapS_pabABC_tu_HFM122 ahdA obtained above by electroporation (Bio-Rad Laboratories, Inc.). The obtained transformed cell solution was spread over LBKm agar medium and then left standing at 30° C. for 2 days.

The obtained colonies were used as a transformant.

### (3) Culture of Transformant

The transformant obtained above was inoculated to 10 mL of CGXII medium (containing 50 μg/mL kanamycin sulfate) shown in Table 3, and cultured at 30° C. for 48 hours. Then, bacterial cells were removed by centrifugation to obtain a culture supernatant. The concentration of 2,5-pyridine dicarboxylic acid in the obtained culture supernatant was quantified in accordance with the method of Reference Example 1.

TABLE 1
CGXII medium
composition (per L)

| | | |
|---|---|---|
| Glucose | 50 | g |
| (NH₄) ₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | ng |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Tryptone | 10 | g |

### (4) Results

As a result of culture of the transformant, 0.40 g/L of 2,5-pyridine dicarboxylic acid was detected in the culture supernatant, demonstrating that 2,5-pyridine dicarboxylic acid can be produced by using this bacterial strain.

### Reference Example 1 Quantification of 2,5-pyridine dicarboxylic acid

2,5-Pyridine dicarboxylic acid was quantified by HPLC. A reaction solution to be subjected to HPLC analysis was appropriately diluted with 0.1% phosphoric acid. Then, insoluble matter was removed using AcroPrep 96-well filter plates (0.2 μm GHP membrane, Nihon Pall Ltd.). The HPLC apparatus used was Chromaster (Hitachi High-Tech Science Corp.). The analytical column used was L-column ODS (4.6 mm I.D.×150 mm, Chemicals Evaluation and Research Institute, Japan). Eluent A was a 0.1% phosphoric acid solution of 0.1 M potassium dihydrogen phosphate, and eluent B was 70% methanol. Gradient elution was performed under conditions involving a flow rate of 1.0 mL/min and a column temperature of 40° C. A UV detector (detection wavelength: 280 nm) was used for the detection of 2,5-pyridine dicarboxylic acid. A concentration calibration curve was prepared using a standard sample [2,5-pyridine dicarboxylic acid (distributor code P0552, Tokyo Chemical Industry Co., Ltd.)]. 2,5-Pyridine dicarboxylic acid was quantified on the basis of the concentration calibration curve.

1. A transformed cell having ability to produce 2,5-pyridine dicarboxylic acids, the transformed cell being derived from a microbe having ability to biosynthesize 4-aminobenzoic acids and having enhanced expression of the following polypeptides (I) and (II):
(I) a polypeptide having 4-aminobenzoic acid hydroxylation activity, and
(II) a polypeptide having 4-amino-3-hydroxybenzoate 2,3-dioxygenase activity.

2. The transformed cell according to claim 1, wherein the (I) polypeptide having 4-aminobenzoic acid hydroxylation activity is (A) a polypeptide which consists of the amino acid sequence represented by SEQ ID NO: 2, or a polypeptide which consists of an amino acid sequence having at least 90% identity to the amino acid sequence represented by SEQ ID NO: 2, and has 4-aminobenzoic acid hydroxylation activity, or (B) a polypeptide which consists of the amino acid sequence represented by SEQ ID NO: 4, or a polypeptide which consists of an amino acid sequence having at least 90% identity to the amino acid sequence represented by SEQ ID NO: 4, and has 4-aminobenzoic acid hydroxylation activity.

3. The transformed cell according to claim 1, wherein the (II) polypeptide having 4-amino-3-hydroxybenzoate 2,3-dioxygenase activity is (C) a polypeptide which consists of the amino acid sequence represented by SEQ ID NO: 6, or a polypeptide which consists of an amino acid sequence having at least 90% identity to the amino acid sequence represented by SEQ ID NO: 6, and has 4-amino-3-hydroxybenzoate 2,3-dioxygenase activity.

4. The transformed cell according to any claim 1, wherein the microbe having the ability to biosynthesize 4-aminobenzoic acids is a microbe having enhanced expression of one or more enzymes selected from the group consisting of the following (III) and (IV):
(III) 4-amino-4-deoxychorismate synthase, and
(IV) 4-amino-4-deoxychorismate lyase.

5. The transformed cell according to claim 1, wherein the transformed cell is E coli or a bacterium of the genus Corynebacterium.

6. A method for producing 2,5-pyridine dicarboxylic acids or salts thereof, comprising a step of culturing the transformed cell according to claim 1.

7. The method according to claim 6, wherein the culture is performed in a culture medium containing a saccharide as a carbon source.

8. The method according to claim 6, further comprising a step of collecting 2,5-pyridine dicarboxylic acids or salts thereof from the culture.

9. The method according to claim 6, wherein the 2,5-pyridine dicarboxylic acids are 2,5-pyridine dicarboxylic acid derivative of the following formula (1): 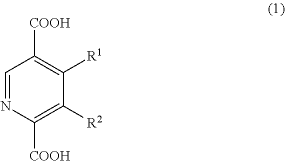
wherein R¹ and R² each independently represent a hydrogen atom, a hydroxy group, a methoxy group, an amino group, a halogen atom, a carboxy group, a methyl group, or an ethyl group, and the 4-aminobenzoic acids are 4-aminobenzoic acid derivatives of the following formula (2): 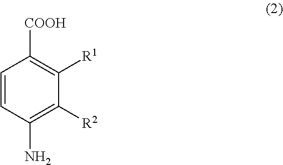
wherein R¹ and R² are as defined above.

10. The transformed cell according to claim 2, wherein the (II) polypeptide having 4-amino-3-hydroxybenzoate 2,3-dioxygenase activity is (C) a polypeptide which consists of the amino acid sequence represented by SEQ ID NO: 6, or a polypeptide which consists of an amino acid sequence having at least 90% identity to the amino acid sequence represented by SEQ ID NO: 6, and has 4-amino-3-hydroxybenzoate 2,3-dioxygenase activity.

11. The transformed cell according to claim 2, wherein the microbe having the ability to biosynthesize 4-aminobenzoic acids is a microbe having enhanced expression of one or more enzymes selected from the group consisting of the following (III) and (IV):
(III) 4-amino-4-deoxychorismate synthase, and
(IV) 4-amino-4-deoxychorismate lyase.

12. The transformed cell according to claim 3, wherein the microbe having the ability to biosynthesize 4-aminobenzoic acids is a microbe having enhanced expression of one or more enzymes selected from the group consisting of the following (III) and (IV):
(III) 4-amino-4-deoxychorismate synthase, and
(IV) 4-amino-4-deoxychorismate lyase.

13. The transformed cell according to claim 4, wherein the transformed cell is E coli or a bacterium of the genus Corynebacterium.

14. A method for producing 2,5-pyridine dicarboxylic acids or salts thereof, comprising a step of culturing the transformed cell according to claim 4.

15. The method according to claim 14, wherein the culture is performed in a culture medium containing a saccharide as a carbon source.

16. The method according to claim 15, further comprising a step of collecting 2,5-pyridine dicarboxylic acids or salts thereof from the culture.

17. The method according to claim 14, wherein the 2,5-pyridine dicarboxylic acids are 2,5-pyridine dicarboxylic acid derivative of the following formula (1): 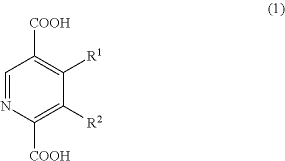
wherein R¹ and R² each independently represent a hydrogen atom, a hydroxy group, a methoxy group, an amino group, a halogen atom, a carboxy group, a methyl group, or an ethyl group, and
the 4-aminobenzoic acids are 4-aminobenzoic acid derivatives of the following formula (2): 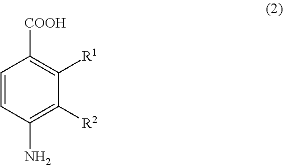
wherein R¹ and R² are as defined above.

18. The transformed cell according to claim 10, wherein the microbe having the ability to biosynthesize 4-aminobenzoic acids is a microbe having enhanced expression of one or more enzymes selected from the group consisting of the following (III) and (IV):
(III) 4-amino-4-deoxychorismate synthase, and
(IV) 4-amino-4-deoxychorismate lyase.

19. The transformed cell according to claim 18, wherein the transformed cell is E coli or a bacterium of the genus Corynebacterium.

20. A method for producing 2,5-pyridine dicarboxylic acids or salts thereof, comprising a step of culturing the transformed cell according to claim 19.


SEQUENCE LISTING (30 sequences)

SEQ ID NO: 1 - DNA, 1176 bp, Caulobacter crescentus
  CDS (1)..(1173): Codon-optimized oligonucleotide
atgcgcactcaggtggctatcgtaggagcaggcccagctggcctgttcttgggccatctcctccgtcaagctggtgtggacgtcgtgattctggaacgcaaagaccgtgcttatgtcgaaggccgagttcgggctggtgtcctggaacgtatcacggtggagctgatggagcgtcttggtgtggatgagcgaatgcgccgagagggcttggtgcatgctggcgctaatcttgcgtctgatggcgagatgttccgtatcgacatggcagagctcacgggtggttccaccgtcatggtttacggccaacaggaggtgatgaaggacctgtttgatgcagcagagcagcgcgatctgcgaattgtctttgacgccgatgcagtgcgtctgcacgatgtggaaggcgaacgtcctcacatcacctggcgcaaagacggggcagaacaccgcctggactgcgatttcattgccggctgcgacggctaccacggagtttctcgtgcgaccattcccgataaggttctcaagaccttcgaacgggtgtatccctttgggtggttgggaatcctggctgaagcacctccgtgtgaccacgagttgatctactcgaaccatgatcgcggttttgccctggcgtcgatgcgctcaccgacacgctcccgctattacgtgcagtgctcactcgacgatcgcctcgaggattggtccgatgaacggttctgggatgaagtttcggttcgcctgggaccggaagcagccgctcggatcgttcgcgcaccttccttcgagaagagcattgccccacttcgctccttcgtttccgagcctatgcggtatggccgccttttcctcgcgggtgatgcggctcatatcgttccacccactggagcgaaagggatgaacttggccgtatcagacgtcatcatgctgtccgaagccctggtcgaacactaccacgaacgctcttccgctggtatcgatggttacagcgcacgtgcacttgcccgcgtctggaaggcggagcgtttcagctggtggtttacctcccttactcaccgcttcccagaccaggacggcttcgaccgcaagatgcaagtcgccgaattggcatacatcaagggttctcgcgctgcccaggtcaccctggcggagaactacgtaggactgccactcgtataa

SEQ ID NO: 2 - protein, 391 aa, Caulobacter crescentus
MRTQVAIVGAGPAGLFLGHLLRQAGVDVVILERKDRAYVEGRVRAGVLERITVELMERLGVDERMRREGLVHAGANLASDGEMFRIDMAELTGGSTVMVYGQQEVMKDLFDAAEQRDLRIVFDADAVRLHDVEGERPHITWRKDGAEHRLDCDFIAGCDGYHGVSRATIPDKVLKTFERVYPFGWLGILAEAPPCDHELIYSNHDRGFALASMRSPTRSRYYVQCSLDDRLEDWSDERFWDEVSVRLGPEAAARIVRAPSFEKSIAPLRSFVSEPMRYGRLFLAGDAAHIVPPTGAKGMNLAVSDVIMLSEALVEHYHERSSAGIDGYSARALARVWKAERFSWWFTSLTHRFPDQDGFDRKMQVAELAYIKGSRAAQVTLAENYVGLPLV

SEQ ID NO: 3 - DNA, 1170 bp, Novosphingobium aromaticivorans
  CDS (1)..(1167): Codon-optimized oligonucleotide
atgaaaacccaggttgccatcattggtgcaggaccagcaggcttgttgctcggtcacttgctcaaagccgaaggaatcgactgcgtggtgctggagcgccaaacgccagactacgtacttggacggattcgcgcgggtgttctggagcagatcaccgtgggtctgatggaacgtcttggcctggatgctcgactgaaggctgagggcctggttgaggagggctttaaccttgccgatggcgaacgcctcattcgcatcgacgtcgctaacttgactggcaagactgtcgtggtgtatggccagaccgagatcaccaaagacttgatggacgctgcacctgaacgtggcctccaggttatctacggtgctagcgaagtggcactgttcgacatcgagtccgatgccccttatgtcacctacgtccatgacggggctcctcgtcgaattgatgcacggttcatcgttgggtgtgacggctttcacggtccgtcacgtaaggcgattccggcttcggtggcccgcgaatacgaacgcgtctatccgtttgggtggctcggcatcctcgcagatgttccaccatgcaatcacgagctgatctacgccaatcacgaacgcggtttcgcgctggcttccatgcgttcccacacgcgtagccgctattacgtagatgttcccctcactgagaaggtggaagattggtctgacgaacgcatttgggacgaactggcagtacgccttggccccgaagcagccgctaacatcacacgaggtccttcgatcgagaagtccatcgctccgcttcggtcctacgtgttcgagccaatgcgccatggttccctgcttctgtgcggagatgcagcgcacattgtcccaccaacaggcgctaaaggcctgaacttggcggcctctgatgtgcactatgcggcagaagcactgaccggattcttcaagcgcgcagataacgatgcagttccgcgttacagcgccaaagcgcttgctcgggtttggaagtctgaacgcttctcctggtcactgaccaagctcatgcatcgcttccctgaggacggaccctttgaacgtgccatgcaagtcgcagagctcgagtacatcgcgacctccaaggctgcgcagacctctatcgccgagaactacgtcggtctgcccgtctaa

SEQ ID NO: 4 - protein, 389 aa, Novosphingobium aromaticivorans
MKTQVAIIGAGPAGLLLGHLLKAEGIDCVVLERQTPDYVLGRIRAGVLEQITVGLMERLGLDARLKAEGLVEEGFNLADGERLIRIDVANLTGKTVVVYGQTEITKDLMDAAPERGLQVIYGASEVALFDIESDAPYVTYVHDGAPRRIDARFIVGCDGFHGPSRKAIPASVAREYERVYPFGWLGILADVPPCNHELIYANHERGFALASMRSHTRSRYYVDVPLTEKVEDWSDERIWDELAVRLGPEAAANITRGPSIEKSIAPLRSYVFEPMRHGSLLLCGDAAHIVPPTGAKGLNLAASDVHYAAEALTGFFKRADNDAVPRYSAKALARVWKSERFSWSLTKLMHRFPEDGPFERAMQVAELEYIATSKAAQTSIAENYVGLPV

SEQ ID NO: 5 - DNA, 528 bp, Bordetella sp.
  CDS (1)..(525): Codon-optimized oligonucleotide
atgatcatcctggaaaacttcaagatgccaaacgtcgacttggaagccgtgatgcgctatcttaccgagactggcaaacgcacccatcagctgtggatggatgatgagactctcgcattcgttgctcgtggtcgggaatatcgctctgagttccacatcaatgcgtcctacgagattcagtactctctgaaaggagcccaagacctcatgtatcgcacacctgaaggcgaagtcaaggtggcacacatgcctgaaggttccgtgttttaccaaccgccattccttccgcattcacctcggtttgcaccggatagcttccagttcatcattgagcgagtacgcaaaccaggggaaatcgacaagttccactggttttgccccaattgcgacaacttcattcacgaggaaaccttctacgtcgacgattaccgcaaggatcccgtttcgcgtgcgtacgacaactacttcaactccctggagtttcgtacgtgcaagaagtgtggcaccgttgctccaggacgcgattaa

SEQ ID NO: 6 - protein, 175 aa, Bordetella sp.
MIILENFKMPNVDLEAVMRYLTETGKRTHQLWMDDETLAFVARGREYRSEFHINASYEIQYSLKGAQDLMYRTPEGEVKVAHMPEGSVFYQPPFLPHSPRFAPDSFQFIIERVRKPGEIDKFHWFCPNCDNFIHEETFYVDDYRKDPVSRAYDNYFNSLEFRTCKKCGTVAPGRD

SEQ ID NO: 7 - DNA, 1863 bp, Corynebacterium glutamicum
atgcgcgttttaattattgataattatgattctttcacgtttaatctcgccacctatgtggaagaggttacgggtcaggcacctgtggtggtgcctaatgatcaagaaatagatgagatgcttttcgacgccgtcatcctctcacctggcccgggccacgccggcgttgcggctgattttggtatctgtgcaggcgtcattgagcgtgcacgcgttccgattttgggtgtgtgtttaggccaccagggcattgcgttggcctatggcggtgatgttgatttggcgcccaggccggtccacggtgaggtttcgcagatcacccatgatggttcaggtttatttgcaggcatccctgaaacgtttgaggcggtgcgttatcactcgatggtggcaacccgcttgccggagtcattgaaagctacagctaccagcgatgatggtttgatcatggcattggcacatgaagtgcttccgcagtggggtgtgcaatttcatccggaatctattggtggacaattcggccatcagatcattaagaacttccttaatttagcgcgcacatatcgctggcaactcacggagaaaactattccgctcagcgttgattcagcagcggtttttgaaacattctttgcccattcctcccatgctttttggctcgatgatgcccaaggaaccagctatcttggtgatgccagcggtcctctcgcacgcacaaaaacccataatgtcggcgagggggatttcttcacctggctaaaggaggatctcgccgccaactcagttgcgcccggtcaaggttttcgtcttggctgggttggttacgttggttatgagcttaaagcggaagctggcgcacgggctgcgcacacttcgagtcttccggatgcgcacctcatttttgccgatcgcgccatcgcagtggaatcggatcaggttcggttgctggcgttgggggagcaggacgagtggtttgaagaaaccatcaagaagctgcataatcttgtcgccccgcggatacctgcgtccggacacctcgctttgcaggttcgagattccaaagatgagtatctcgacaaaattcgcagagcccaggagctgattactcgcggcgaatcgtatgaaatctgcctgaccacaaaacttcagggcaccactgatgtggcccctctggctgcctatctagcactgcgtggggccaatcccaccgcatatggtgcgtatcttcagctgggggatacctctattttgagttcctcgccggagcggttcatcaccattgattcggcagggtatgtggaatcaaagcccattaaaggcaccaggccgcgtgggcgaacagcgcaagaagaccaagaaatcattgctgagctgcgcagtaatcctaaagatcgtgcagaaaacttgatgatcgtggatttggtccgcaacgacttagcccgcggcgctttgcccaccacagttaaaacatccaagcttttcgacgtcgaaacctacgccacagtccaccaacttgtcagcaccgtctctgcagagttggggccacgcagtccgattgagtgcgtgcgcgcagcattccccggtggttcgatgactggtgccccaaagctgcgcaccatggagatcatcgatgagctggaggcagctcctcgcggtatttactcaggtggcttgggatatttttccctcgacggcgcagttgatctctccatggtgatcagaactctcgtcatccagaacaatcacgtggagtacggagtgggcggtgcacttcttgctctgtctgatccggaggctgagtgggaggaaatccgcgttaaatcacggcctctgctgaatttgtttggggttgaattcccatga

SEQ ID NO: 8 - DNA, 726 bp, Corynebacterium glutamicum
atgacgtacctcgtgtgggacggtgcaacactcgtagaaggcgcgctggaatcaacacccacagttgttgattcctacctagccaaagaccaccgcgtggtgcgctgggatcttcatgaacagcgcttcgccactagcgtggacgtggacccgtgggattttctccacgcagtaagggaagcaattccacgccagggctcatggtttcccaaagttgaatggcatggcgatgatcttttcgcagtcaatattcgcccggcaccaacactgcgaaaggccacatcattgtggctttccgaagacccagatccacgcacacagccaaccattaaaggcccagacctagatgtgcttgctcaccttcgcagtcgcgccaacgataacggctgcgatgatgcgctgttgatcagcgcggatgggttcattctggaagctgccaacgccaccgtggtgttttgggcggatccacagacggtcatcgtgcccaggggagatgtgctcccatcggtgacactcgccgcaaccattccgctgtgggaaaaagccggaatcacattgcgctatcaaaacattcggcacattggttttcccgcgtggtgcggtagttcgctgcatggttggacacctgtggtcagttggggcaggggattgggcaaaattgcagcagcgaaagctccatcggtgaagccctggaatgaaaaattgcgcccaaccatttttctgtaa

SEQ ID NO: 9 - DNA, 42 bp, Artificial Sequence
  Synthetic oligonucleotide
tattaattaaatgcgcgttttaattattgataattatgattc

SEQ ID NO: 10 - DNA, 45 bp, Artificial Sequence
  Synthetic oligonucleotide
ttgcggccgcttgtttaaacctccttacagaaaaatggttgggcg

SEQ ID NO: 11 - DNA, 33 bp, Artificial Sequence
  Synthetic oligonucleotide
aaatttaaacctcctttacagaaaaatggttgg

SEQ ID NO: 12 - DNA, 28 bp, Artificial Sequence
  Synthetic oligonucleotide
ggaggtttaaacaagcggccgcgatatc

SEQ ID NO: 13 - DNA, 35 bp, Artificial Sequence
  Synthetic oligonucleotide
aggaggtttaaatttatgcgcactcaggtggctat

SEQ ID NO: 14 - DNA, 35 bp, Artificial Sequence
  Synthetic oligonucleotide
cttgtttaaacctccttatacgagtggcagtccta

SEQ ID NO: 15 - DNA, 31 bp, Artificial Sequence
  Synthetic oligonucleotide
gctatcaaaacattcggcacattggttttcc

SEQ ID NO: 16 - DNA, 29 bp, Artificial Sequence
  Synthetic oligonucleotide
ggaagatgcgtgatctgatccttcaactc

SEQ ID NO: 17 - DNA, 24 bp, Artificial Sequence
  Synthetic oligonucleotide
ttacagaaaaatggttgggcgcaa

SEQ ID NO: 18 - DNA, 22 bp, Artificial Sequence
  Synthetic oligonucleotide
atgcgcactcaggtggctatcg

SEQ ID NO: 19 - DNA, 342 bp, Artificial Sequence
  Synthetic oligonucleotide
tacgtacctgcaggtagcgtgtcagtaggcgcgtagggtaagtggggtagcggcttgttagatatcttgaaatcggctttcaacagcattgatttcgatgtatttagctggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaaaggaggatctaaattatgaataatataaaaggaggaattaattaa

SEQ ID NO: 20 - DNA, 36 bp, Artificial Sequence
  Synthetic oligonucleotide
accatttttctgtaatacgtacctgcaggtagcgtg

SEQ ID NO: 21 - DNA, 35 bp, Artificial Sequence
  Synthetic oligonucleotide
cacctgagtgcgcatttaattaattcctcctttta

SEQ ID NO: 22 - DNA, 29 bp, Artificial Sequence
  Synthetic oligonucleotide
gcttgttagatatcttgaaatcggctttc

SEQ ID NO: 23 - DNA, 18 bp, Artificial Sequence
  Synthetic oligonucleotide
ggaggtttaaacaagcgg

SEQ ID NO: 24 - DNA, 27 bp, Artificial Sequence
  Synthetic oligonucleotide
aatttagatcctcctttggacttcgtg

SEQ ID NO: 25 - DNA, 36 bp, Artificial Sequence
  Synthetic oligonucleotide
aggaggatctaaattatgcgcactcaggtggctatc

SEQ ID NO: 26 - DNA, 37 bp, Artificial Sequence
  Synthetic oligonucleotide
cttgtttaaacctccttatacgagtggcagtcctacg

SEQ ID NO: 27 - DNA, 39 bp, Artificial Sequence
  Synthetic oligonucleotide
cgcttgtttaaacctccttatacgagtggcagtcctacg

SEQ ID NO: 28 - DNA, 30 bp, Artificial Sequence
  Synthetic oligonucleotide
gccgcgatatcgttgtaaaaaaccccgctc

SEQ ID NO: 29 - DNA, 42 bp, Artificial Sequence
  Synthetic oligonucleotide
aggtttaaacaagcgatgatcatcctggaaaacttcaagatg

SEQ ID NO: 30 - DNA, 36 bp, Artificial Sequence
  Synthetic oligonucleotide
caacgatatcgcggcttaatcgcgtcctggagcaac